(19) United States
(12) Patent Application Publication
(10) Pub. No.: US 2003/0104039 A1
(43) Pub. Date: Jun. 5, 2003

(54) Wound dressing with a reduced accretion tendency

(76) Inventors: Achim Berthold, Andernach (DE); Walter Mueller, Neuwied (DE)

Correspondence Address: BIRCH STEWART KOLASCH & BIRCH, PO BOX 747, FALLS CHURCH, VA, 22040-0747, US

(21) Appl. No.: 10/239,161

(22) PCT Filed: Mar. 8, 2001
(86) PCT No.: PCT/EP01/02600

(30) Foreign Application Priority Data
Mar. 23, 2000 (DE) 100 14 557.4

(51) Int. Cl. A61L 15/00; A61L 15/16

(52) U.S. Cl. 424/445

(57) ABSTRACT

A wound dressing with reduced accretion tendency and comprising a layer (2) which is at least single-ply, is characterized in that said layer is connected to a porous membrane or semi-permeable film (1) which faces the wound.

The invention relates to a wound dressing for promoting wound healing, which stands out for showing no or only little tendency for accretion with the wound.

Wound dressings have to fulfil a variety of requirements in order to promote wound healing in the best possible way. For one thing, the wound must be protected from environmental influences, such as intrusion of foreign bodies and exertion of pressure from the outside. For this reason, wound dressings have to be germ-free and sterilisable in order to reduce the likelihood of secondary infection. Furthermore, wound dressings serve to maintain the wound warm and moist, as wounds heal better when maintained moist under occlusion. Inter alia, the following mechanism is being discussed in the literature as the cause for the “healing” effect of an occlusive environment: Due to the absence of oxygen, the wound is forced to bring oxygen into the wound area via the blood. This happens by increased pannus formation under vascularisation and thereby enhanced wound healing.

On the other hand, the wound dressing is to exhibit good absorptive power and sufficient absorptive capacity in order to permit the wound exudate to draw off and in order to prevent the formation of moist chambers. In particular, wound exudate which does not drain off and is possibly infected with germs carries a high risk of infection.

A wound dressing, in addition, is to possess sufficient wet fastness and is to prevent that residues remain, which could irritate wound healing. To promote the wound healing process, a wound dressing should ensure sufficient oxygen and water vapour permeability, and thereby breathability. It should conform to the wound in order to enable a constant uptake of wound exudate and, possibly, release of active substances, be skin-friendly, as well as easy to apply and remove, to ensure wound rest.

With the known wound dressings, it has frequently been found that these have a disadvantageous tendency to adhere to conglutinate with the wound. The reason for this is that the newly formed tissue, which has been formed in the course of wound healing, e.g. granulation tissue, blood vessels, etc, grows together with the bottom side of the wound dressing, with cells migrating into the wound dressing or forming branches which bud into the wound dressing. Since with every change of bandage, which involves taking off the conglutinated bandage, fresh granulation tissue and epithelial edges are torn off along with it, healing is in this way retarded by such conglutinations of the wound dressing with the wound.

It is true that the risk of adhesion or accretion of the wound with the wound dressing can be reduced by changing the bandage frequently, so that, due to the short time interval, adhesion or accretion can not occur. It is, however, preferable to change the wound dressing only rarely or less frequently, so as to cause as little disturbance to the healing process as possible. Since taking off a completely soaked wound dressing causes a loss in wound exudate components and thereby of immunocompetent cells (these are cells which recognize antigens and produce the corresponding antibodies, and cells which are capable of receiving, and thus neutralizing, foreign bodies), the healing process is interrupted or disturbed with each change of wound dressing.

It was thus the object of the present invention to provide a wound dressing which has only little or no tendency towards growing together with the wound. At the same time, such wound dressing is to meet the above-mentioned general requirements.

Surprisingly, this object is achieved in that, in a wound dressing comprising a layer which is at least single-ply, said layer is connected with a porous membrane or a semi-permeable film on the side facing the wound and coming into contact with the wound. This porous membrane or film, which is located between the wound surface and the, at least single-ply, layer of the wound dressing, inhibits or prevents ingrowth or budding of cells into the film and into the layer on top of the film. Thereby, accretions of the healing wound with the wound dressing are prevented.

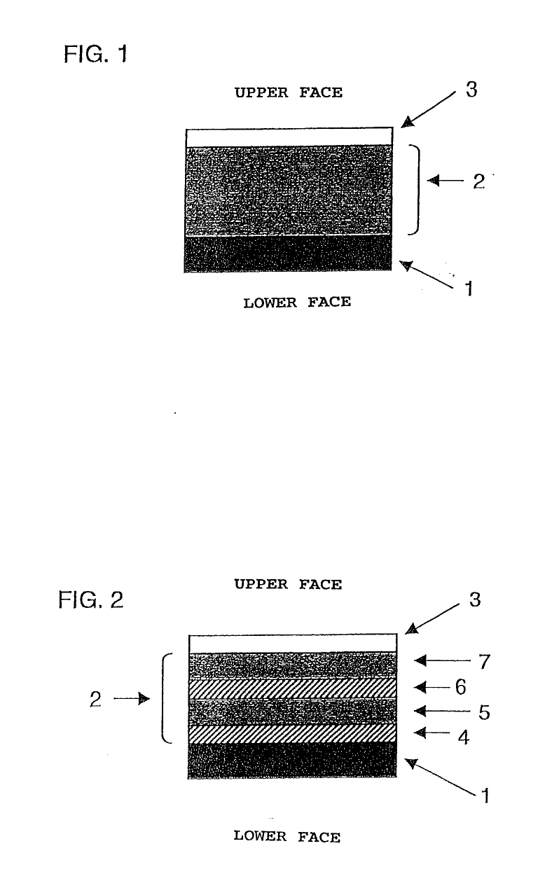

In the following, the wound dressing according to the present invention will be explained in more detail by way of the embodiment examples shown in FIGS. 1 and 2. FIGS. 1 and 2 represent the inventive wound dressings in cross-section, to show the layer structure thereof.

The wound dressing with reduced accretion tendency according to the present invention comprises, in a simple embodiment, as shown in FIG. 1, a porous membrane or semi-permeable film (1) and a superimposed layer (2). The membrane or film (1) forms the lower side of the wound dressing, which lower side will be placed on the wound surface. The layer (2) forms the outer layer or upper side of the wound surface.

Basically, those membranes or films are suitable as a porous membrane or semi-permeable film that are commonly used in microfiltration or ultrafiltration. Preferably, the pore diameter or exclusion limit is selected such that tissue cells can no longer grow into the membrane or film. Especially suitable are membranes the pore size of which is smaller than 10 μm, usefully smaller than 1 μm, and preferably smaller than 0.25 μm.

Semi-permeable films are understood to be those films which are permeable to water and to substances dissolved therein, including protein molecules. The usable membranes or films may, for instance, be manufactured on the basis of cellulose acetate, cellulose triacetate, cellulose nitrate, regenerated cellulose, polyvinyl alcohol or polyamide.

The layer (2) which is located atop the membrane or film, and is preferably fixedly connected thereto, is preferably configured as an absorptive layer which serves to take up and drain wound exudate. It may therefore contain absorptive materials which are known to those skilled in the art. Examples of suitable materials are swellable polymers, e.g. hydrogels based on gelatine, collagen, collagen-heparin complexes, alginates, pectins, starches, albumin, agarose, types of cellulose (e.g. carboxymethyl-cellulose, methylcellulose, hydroxypropylmethyl cellulose), polyethylene glycolene, polyvinyl pyrrolidone, methyl-pyrrolidone chitosan, cyclo-dextrines, hyaluronic acids, polyanhydrides, polyvinyl alcohols, polyacrylic acid, poloxamers and polyacrylamides.

The outer or upper side of the wound dressing may additionally be provided with a cover layer (3) which rests on the layer (2), respectively is connected therewith. It may serve, for instance, as a protective layer giving strength to the wound dressing and protecting it from becoming damaged. It may also be moisture-impermeable or water vapour-impermeable.

The structure of the inventive wound dressing in addition comprises an active substance-impermeable cover layer as well as, optionally, a likewise active substance-impermeable protective film or strippable film. Suitable as a cover layer is first of all polyester, but also almost any other skin-friendly plastics such as polyvinyl chloride, ethylene-vinylacetate copolymer, polyvinyl acetate, polyethylene, polypropylene, polyurethane, cellulose derivatives and many others more. In the individual case, the cover layer may be provided with an additional layer, e.g. by vapour-deposition of metals, especially aluminium, or other diffusion-blocking additives, such as silicon dioxide or like substances known to those skilled in the art. For the detachable protective layer, the same materials may be used as are used for the backing layer, provided that it is rendered detachable by a suitable surface treatment such as siliconisation. But other detachable protective layers such as polytetrafluoro-ethylene-treated paper, cellophane®, polyvinyl chloride or the like may be used as well.

Especially advantageous are those embodiments of the wound dressing according to this invention where either the membrane or film (1) and/or the layer (2) contain a wound healing-promoting pharmaceutical active agent—or several agents—which can be delivered to the wound by or via the membrane or film. For example, the outer side of the membrane or film (1), which faces the wound, may be loaded with active substance(s), or the layer (2) may be soaked or impregnated with one or more active substances. Preferably, active substances which promote wound healing are used for this purpose.

Where the wound dressing contains active substances, it is especially useful if at the same time it comprises agents which enable a controlled release of the active substance or substances. This can be accomplished, for instance, with the aid of additional membranes controlling the release of active substance, or by using polymer matrices which serve as active substance reservoir and from which the active substance is released in a controlled manner.

As wound healing-promoting active substances which may be incorporated in the wound dressing according to the invention, those selected from the group of the growth factors, the antibiotics, the antiseptics, the anti-mycotics, the analgesics, the enzyme inhibitors, the antihistaminics, the vitamins, the glucocorticoids, the antiviral active agents, the steroids, the nucleosides, including the deoxyribonucleosides, the enzymes (e.g. protein kinase C), as well as those selected from the group of the hormones, are especially suitable. Of the group of the growth factors, the following are particularly suitable: platelet derived growth factor, epidermal growth factor, platelet derived endothelial cell growth factor, acidic fibroblast growth factor, basic fibroblast growth factor, transforming growth factor alpha, transforming growth factor beta, keratinocyte growth factor, insulin-like growth factor 1, insulin-like growth factor 2, and tumour necrosis factor; among the growth factors, the platelet derived growth factor is particularly preferred.

From the group of the hormones, insulin, growth hormones, tyroxin, triiodine thyronine are preferably selected.

As can be seen from FIG. 2, the invention also embraces embodiments where the layer (2) is made up of two or more plies. For instance, this layer may consist of four layers (4-7) of which, for example, two layers (5, 7) are polymer-containing and the respective two other layers (4, 6) are made up of a nonwoven-like or a woven fabric-like material. Likewise, the layer (2) may be comprised of two individual plies, of which one is manufactured on the basis of a polymer, and the other consists of a nonwoven-like or woven fabric-like material. However, a large number of further embodiments is also possible which may result from the combination and arrangement of suitable materials in various plies of the layer (2).

The embodiment shown in FIG. 2 likewise possesses, in accordance with the invention, a porous membrane or semi-permeable film (1) at the bottom side, which bottom side faces the wound, so as to inhibit adhesion of the wound dressing on the wound. In addition, such embodiment may be provided with a cover layer (3) on the upper side, which upper side is averted from the wound.

If an embodiment is selected wherein the layer (2) is of a double-ply or multiple-ply structure and contains active substance(s), this results in the additional possibility of configuring only one or only several of these plies so as to be active substance-containing. It is also possible, for example, to provide one of the plies as a control membrane which regulates the active substance release from an active substance-containing ply located thereabove.

The wound dressing with reduced accretion tendency according to the invention is suitable for general use in wound toilet. It is particularly advantageous to use the wound dressing for treating chronic or slow-healing wounds.

Suitably, for storage purposes, the wound dressing according to the invention is in addition provided with a strippable film at the outer side of the film or membrane, which strippable film may consist, for instance, of the materials mentioned with respect to the cover layer, and/or the wound dressing is contained in a surrounding outer package of usual material.

1. Wound dressing, comprising a layer (2) which is at least single-ply, characterized in that said layer has, on the wound-contact side thereof, a porous membrane or semi-permeable film (1) which prevents ingrowth or budding of cells into the membrane or film and the layer, said porous membrane or semi-permeable film (1) containing at least one wound healing-promoting active substance, and preferably only the outer side, which faces the wound, being loaded with the active substance(s).

2. Wound dressing according to claim 1, characterized in that the layer (2) is at least partially made up of absorptive materials or contains such materials.

3. Wound dressing according to claim 1 or 2, characterized in that the pore diameter of the porous membrane is selected such that intrusion or ingrowth of tissue cells into the membrane is prevented, the pore diameter being below 10 μm, usefully below 1 μm, and preferably below 0.25 μm.

4. Wound dressing according to one or more of the preceding claims, characterized in that the layer (2) is provided with a cover layer (3) on the external side, which is averted from the skin, which cover layer (3) forms the outermost layer of the wound dressing.

5. Wound dressing according to one or more of the preceding claims, characterized in that the layer (2) comprises two or more plies, preferably with at least one ply being made up of nonwoven-like material or woven fabric-like material, and at least one further ply being made up of polymers.

6. Wound dressing according to one or more of the preceding claims, characterized in that the layer (2) contains at least one active substance which promotes wound healing.

7. Wound dressing according to one or more of the preceding claims, characterized in that a growth factor, in particular platelet derived growth factor, is added as an active substance promoting wound healing.

8. Wound dressing according to one or more of the claims, characterized in that it comprises means which effect a controlled release of the active substance(s), said means preferably being a release-controlling membrane or a release-controlling polymer composition.

9. Wound dressing according to one or more of the preceding claims, characterized in that it is, in addition, provided with a protective film on the outer side of the membrane or film, or that it is contained in a surrounding outer package.

10. Use of the wound dressing according to one or more of the preceding claims for wound treatment and promoting wound healing, preferably for longer-term treatment of chronic or slow-healing wounds.
